(19) United States
(12) Patent Application Publication
(10) Pub. No.: US 2024/0261160 A1
(43) Pub. Date: Aug. 8, 2024

(54) CASE FOR HYGIENIC TAMPONS

(71) Applicant: AGNIESZKA PRZYBORSKA-BO-JANOWSKA, Banino (PL)

(72) Inventor: Joanna Michalak, Banino (PL)

(21) Appl. No.: 18/567,047

(22) PCT Filed: Jun. 3, 2022
(86) PCT No.: PCT/PL2022/000036

§ 371 Date: Dec. 5, 2023

(30) Foreign Application Priority Data
Jun. 5, 2021 (PL) P.438072

(51) Int. Cl. A61F 13/551; A01N 25/12; A01N 59/06; A01N 59/16; A01N 59/20; A45C 11/00; A61F 13/84

(52) U.S. Cl. CPC A61F 13/55175; A01N 25/12; A01N 59/06; A01N 59/16; A01N 59/20; A45C 11/008; A61F 13/8405

(57) ABSTRACT

A case (1) for hygienic tampons including an elongated housing (2) inside which a gutter-shaped rail (3) with at least one slot (4) for a single hygienic tampon is placed. Each tampon slot (4) in the rail (3) is provided with an opening that makes it easier to remove the tampon by inserting a finger and pushing the tampon out of it. A plug (5) with a handle is attached to the rail (3).

### CROSS REFERENCE TO RELATED APPLICATION

This application is a national stage entry of PCT/PL2022/000036 filed Jun. 3, 2022, under the International Convention and claiming priority over Poland Patent Application No. P.438072 filed Jun. 5, 2021.

### FIELD OF THE INVENTION

The present invention constitutes a refillable case for menstrual hygiene tampons.

### BACKGROUND OF THE INVENTION

Hygiene products belong to the family of everyday objects that female users carry with them whenever there might arise a necessity to use them. The problem is the hygienic storage of these products in hand luggage, after opening the commercial packaging. Hygienic cases for repeated use are already available, but their construction does not always provide adequate hygiene protection for these products. Hygienic materials are often exposed to the influence of moisture, which favors the development of dangerous bacterial flora inside the case.

The package for sanitary products, in particular sanitary napkins and panty liners, is known from the international patent application WO202020193565, comprising at least one container for receiving a plurality of sanitary products, each container having an opening through which a sanitary product can be placed in a respective container and removed from it. In order to protect hygiene products from soiling and to keep them together when several hygiene products are stored in the packaging bag, at least one closure flap is provided which closes the opening of at least one container.

A sanitary case for sanitary napkins, panty liners and tampons, usually rectangular in shape and having three compartments, a top, middle and bottom is known from patent publication No. CA2109782. The case is generally rectangular in shape and has flaps on the sides, thus forming a compartment. The compartment comprises an outer surface supporting two pockets, thus forming compartments the openings of which are oriented opposite to each other. The case is secured with Velcro.

A multiple applicator is known from the international patent application WO2005049222. The applicator has an elongated housing that is divided into a plurality of closed chambers. The longitudinal housing is cut into shorter lengths. The removed portion of the elongated housing serves to apply the liquid or sticky substance contained therein and is removed after application. The remainder of the elongated housing with the remaining unused applicator tips and fluid is retained for subsequent use.

Plastics are widely used in many areas of life, from everyday household items, through packaging and housings of many devices, to fabrics or building materials. A special feature of plastics is their durability, and the necessary properties of plastic products are designed by the appropriate selection of the polymer itself and by the composition of functional additives whose task is to provide new features to the polymer or to strengthen the already existing properties of the plastic.

A well-known and more and more commonly used method of hygienisation is the use of biologically active agents deposited on the surface. Metals or metal oxides in particular have a biocidal effect.

Patent DE19750128 discloses a process for treating metal or alloy surfaces in order to impart an effective biocidal effect. The process involves coating the surface with a layer containing metallic copper. The method is used especially in air conditioners—and particularly in car air conditioners, in which the metal surfaces are covered with metallic copper with a bactericidal effect.

Metal and metal oxide nanoparticles are becoming increasingly popular as biocides. The only limitation in the use of nanoparticles is the problem of overcoming the limitations of biocide application.

The European patent EP 2244973 describes a powder consisting of metallic silver nanoparticles deposited on a silica bed, its preparation and application. A powder comprising silica bed grains containing nano metallic silver particles, wherein the diameter of at least 95% of the silica support grains are in the range 450 to 650 nm and the diameter of at least 95% of the nano metallic silver particles are in the range 0 to 20 nm. The powder can be used to prepare bioactive materials selected from the group consisting of dispersion, emulsion, layered, textile, fibrous or polymerized materials.

The materials obtained are used for the production of fabrics, paints, plastics, paper products, insulation materials, furniture industry products, pharmaceuticals, food or cosmetic products.

Patent PL226866 describes aground heat exchanger, the air channels of which are made of polyethylene or polypropylene, with an admixture of aluminum powder in the amount of 0.01 to 10%, and are covered on the inside with antiviral, antibacterial and antifungal properties with a coating containing silver nitrate, hydrogen peroxide, ethanol, isopropanol, and potassium nitrate.

The patent CN100536661 discloses a nanoparticle air-purifying bed. The bed contains ZnO and Fe₂O₃ nanoparticles and sodium zirconium phosphate grains. The grain of the material is 0.5 to 3 mm. The method of preparation includes the steps of adding nanoparticles of ZnO, Fe₂O₃ and sodium zirconium phosphate to the container; next, the mixture is uniformly mixed and grains are made with a disc granulator with granulation controlled in the range of 0.5 to 3 mm and the drying temperature to be controlled between 110 and 130° C.; quick screening and packing of the grains after drying.

Patent application DE102005032616 describes molding plastics with improved mechanical properties. The material has a UV-cured coating containing a polyacrylate, an inorganic oxide filler, a polyol monoacrylate ester, a vinyl silane, a photoinitiator, and an antimicrobial agent in the form of metallic silver or silver salt.

### SUMMARY OF THE INVENTION

The essence of the invention is that the sanitary pad case (1) consists of an elongated casing (2) with a gutter-shaped rail (3) inside, with at least one slot (4) for a single sanitary tampon. Each tampon slot (4) in the rail (3) is provided with an opening that makes it easier to remove the tampon, by inserting a finger and pushing the tampon out of it. A plug (5) equipped with a handle (6) is attached to the rail (3). Preferably, the plug (5) is equipped with protrusions (7) constituting an element of the lock securing the rail against spontaneous falling out of the housing (2) of the case (1) after placing the plug inside the housing (2). In case of more tampon slots (4) in the rail (3), they are to be arranged longitudinally along the longitudinal axis of the rail (3) and parallel to the longitudinal axis of the housing (2).

### BRIEF DESCRIPTION OF THE DRAWINGS

FIG. 1 shows a refillable case for menstrual hygiene tampons according to the present invention having a cylindrical shape;

FIG. 2 shows the inner elements of a case of FIG. 1; and

FIG. 3 shows the case of FIG. 1 having a rectangular shape.

### DETAILED DESCRIPTION OF THE INVENTION

The inner face of the handle (6) and the inner face of the casing (2) are flat and parallel to each other, ensuring tightness after placing the plug (5) inside the casing (2). Preferably, the housing (2) should have a cylindrical shape with a circular end cap (8) and a cylindrical end cap (5). Another advantageous solution is for the housing (2) to have a rectangular shape with an orthogonal plug (8) and a rectangular plug (5). The housing (2), the rail (3) with slots (4) and the plug (5) are made of a plastic composite, preferably a thermoplastic, especially polyethylene or polypropylene, which, in addition to the optional standard components of the composite, contains from 10 ppm to 800 ppm of a biologically active substance in the form of nanoparticles selected from silver, copper, zinc, aluminum and iron Ill oxide nanoparticles or mixed in any proportions.

The design of the case ensures convenient storage in hand luggage, tightness against possible moisture and hygiene of the materials stored within. The variant containing a stock of several hygiene items, by placing them one after the other, allows only one compartment containing the tampon to be exposed, while the others are still hidden inside the housing.

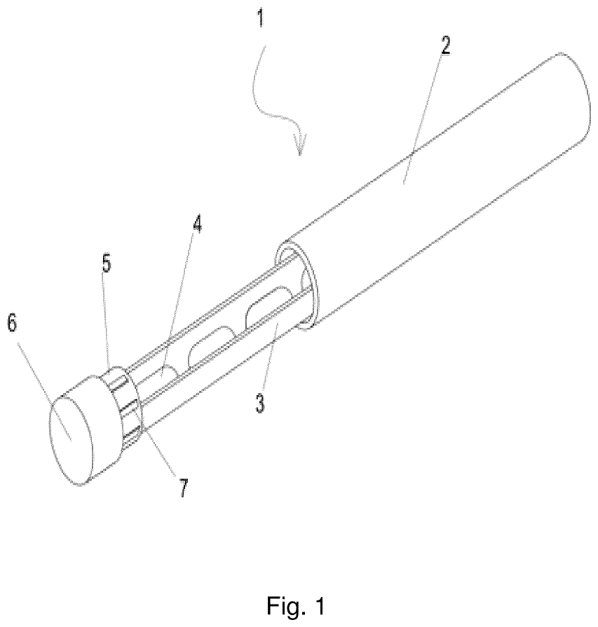

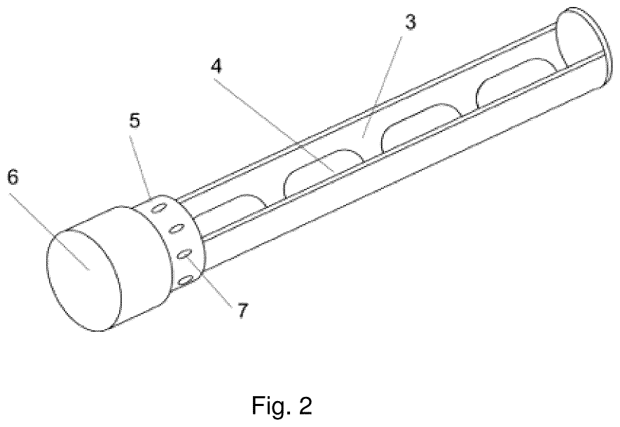

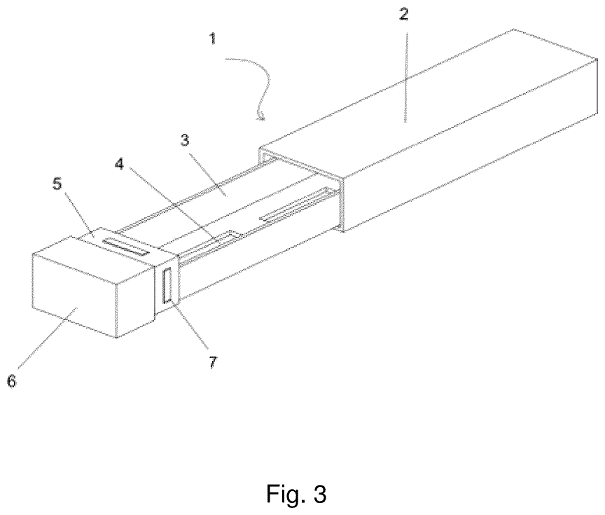

The subject of the invention is presented in the examples of manufacture and in the drawing, in which FIG. 1 shows a cylindrical case, FIG. 2 the inner elements of a case, FIG. 3 a rectangular case.

### Example I

The case (1) for hygienic tampons consists of a cylindrical, elongated casing (2), terminated with a plug (8), with a rail inside (3), with three slots (4) for hygienic tampons, located longitudinally along the longitudinal axis of the rail (3) and parallel to the longitudinal axis of the housing (2). The rail (3) is fitted with a cylindrical plug (5) equipped with a handle (6), with protrusions (7) ensuring firm closure of the case (1) after placing the plug inside the housing (2). The housing (2), the rail (3) with the slots (4) and the plug (5) are made of polyethylene, which contains 10 ppm of silver nanoparticles.

### Example II

The case (1) for hygienic tampons consists of an elongated rectangular casing (2), terminated with a plug (8), inside which a rail (3) is placed with two slots (4) for hygienic tampons, located longitudinally along the longitudinal axis of the rail (3) and parallel to the longitudinal axis of the housing (2). Affixed to the rail (3) is a rectangular plug (5), equipped with a handle (6) and protrusions (7), ensuring firm closure of the case (1) after placing the plug inside the housing (2). The housing (2), the rail (3) with the slots (4) and the plug (5) are made of polypropylene, particularly polyethylene or polypropylene, which contains 800 ppm of silver, copper, zinc, aluminum and iron oxide Ill nanoparticles, mixed in weight proportions 1:1:1:1:1.

### Example III

The case (1) for hygienic tampons consists of a cylindrical, elongated casing (2), terminating with a plug (8), inside which a rail is mounted (3) with three slots (4) for hygienic tampons located longitudinally along the longitudinal axis of the rail (3) and parallel to the longitudinal axis of the housing (2). The rail (3) is fitted with a cylindrical plug (5) equipped with a handle (6) and with protrusions (7) ensuring firm closure of the case (1) after placing the plug inside the housing (2). The housing (2), the rail (3) with the slots (4) and the plug (5) are made of polystyrene, which contains 300 ppm of aluminum nanoparticles.

1. A case for hygienic tampons made of plastic comprising:
an elongated housing (2) having, inside a gutter-shaped rail (3), with at least one slot (4) for a single hygienic tampon, wherein each one of the tampon slot (4) in the rail (3) includes a hole, and a plug (5) with a handle (6) is attached to the rail (3);
wherein when there are more slots (4) in the rail (3), the slots are situated longitudinally, along a longitudinal axis of the rail (3) and parallel to a longitudinal axis of the housing (2), with an inner face of the handle (6) and
wherein an inner face of the housing (2) is flat and parallel to each other ensuring tightness after inserting the plug (5) inside the housing (2);
wherein the housing (2), the rail (3) with slots (4) and the plug (5) are made of a plastic composition which, apart from possible standard components of the composite, contains from 10 ppm to 800 ppm of active biological substances in form of nanoparticles, selected from nanoparticles of silver, copper, zinc, aluminum, iron III oxide, or combination thereof.

2. The case according to claim 1, wherein the plug (5) includes protrusions (7) constituting an element of the lock protecting the rail against falling out of the housing (2) of the case (1) after placing the plug inside the housing (2).

3. The case according to claim 1, wherein the housing (2) has a cylindrical shape with a circular end plug (8), and a cylindrical end plug (5).

4. The case according to claim 1, wherein the housing (2) has a rectangular shape with an orthogonal end plug (8), and a rectangular end plug (5).

5. The case according to claim 1, wherein the case is made of a thermoplastic selected from the group consisting of polyethylene and polypropylene.
